(19) United States
(12) Patent Application Publication
(10) Pub. No.: US 2007/0111952 A1
(43) Pub. Date: May 17, 2007

(54) Glycosylated triketide delta lactones

(76) Inventors: Benoit Mathieu, Bruxelles (BE); Edmond De Hoffmann, Louvain-La-Neuve (BE); Charles Van Hove, Ceroux-Mousty (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/580,930

(22) PCT Filed: Nov. 28, 2003
(86) PCT No.: PCT/EP03/13406

§ 371 Date: May 30, 2006

(51) Int. Cl. A61K 31/7048

(52) U.S. Cl. 514/25; 536/17.1

(57) ABSTRACT

The present invention relates to compounds of formula (1) and salts, stereoisomeric forms, racemic mixtures, prodrugs, esters and metabolites thereof. R₁ is a glycosyl moiety, hydroxyl-protected acetate derivatives thereof or amino derivatives thereof; R₂, R₃ and R₄ are, each independently, selected from hydrogen, C₁₋₆alkyl, C₂₋₂₀alkenyl, C₆₋₂₀arylalkyl, C₃₋₇cycloalkyl, C₃₋₇cycloalkylC₁₋₆alkyl, C₆₋₂₀aryl aralkyl, akylcarbonyloxy, arylcarbonyloxy, alkyloxy, alklylthio, alkylamino, alkyloxyalkyl, alkanoyl, alkylcarbonylalkyl, optionally substituted by one or more substituents independently selected from alkyl, aralkyl, aryl, cycloalkyl, alkyloxycarbonyl, carboxyl, aminocarbonyl, hydroxy, cyano, halogen or amino optionally mono- or disubstituted wherein the substituents are independently selected from alkyl, aryl, aralkyl, aryloxy, arylamino, aryloxyalkyl, arylaminoalkyl, aralkoxy, and alkoxy.

### FIELD OF THE INVENTION

The present invention relates to novel glycosylated triketide delta lactone compounds which have antimicrobial activities, to pharmaceutical composition comprising the same, and to therapeutic uses thereof.

### BACKGROUND OF THE INVENTION

Polyketides represent a large family of diverse compounds synthesized in general from 3-carbon units through a series of Claisen-type condensations and subsequent modifications. Members of this group include antibiotics such as tetracyclines, anticancer agents such as daunomycin, and immunosuppressants such as FK506 and rapamycin. Polyketides occur in many types of organisms including fungi and mycelial bacteria, in particular, the actinomycetes.

The polyketides are synthesized by polyketide synthases (PKS). This group of enzymatically active proteins is considered in a different category from the fatty acid synthases which catalyze condensation of 2-carbon units to result in, for example, fatty acids and prostaglandins. Type I PKS are large, multifunctional enzymes that consist of several discrete modules, each responsible for one round of acyl chain elongation. A typical module is comprised of acyltransferase (AT), acyl carrier protein (ACP), and ketosynthase (KS) domains catalyzing each decarboxylative condensation. Modules may also contain ketoreductase (KR), dehydratase (DH) and enoyl reductase (ER) domains which determine the level of processing of the β-keto group after each extension step. The C-terminus of the last module typically contains a thioesterase (TE) domain that catalyses acyl chain release and lactonization.

Manipulation of PKS systems have allowed the production of triketide lactones, the synthesis of which has been extensively studied to understand the biosynthesis of polyketides. No biological activity has been reported for these triketide lactones.

In view of the foregoing, it is a main object of the present invention to provide novel triketide delta lactone compounds that are highly active against a number of microorganisms in human being, plants and animals. Another object of the present invention is to provide a process for the preparation of said compounds. A further object of the present invention is to provide a pharmaceutical composition comprising, as an active ingredient, said compound or a pharmaceutically acceptable salt thereof. Still further object of the present invention is to provide a method for treating or preventing infectious diseases caused by microorganisms, which comprises administering said compound to human being, plants or animals.

### SUMMARY OF THE INVENTION

It was surprisingly found with the present invention that the glycosylated triketide δ-lactones of formula (1) had biostatic and biocide activities.

In a first aspect, the present invention provides novel compounds of formula (1) and salts, stereoisomeric forms, racemic mixtures, prodrugs, esters and metabolites thereof, wherein 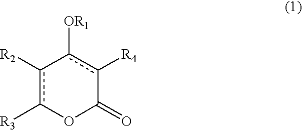
R₁ is a glycosyl moiety, hydroxyl-protected acetate derivatives thereof or amino derivatives thereof;
R₂, R₃ an R₄ are, each independently, selected from the group comprising hydrogen, C₁₋₆alkyl, C₂₋₂₀alkenyl, C₆₋₂₀arylalkyl, C₃₋₇cycloalkyl, C₃₋₇cycloalkylC₁₋₆alkyl, C₆₋₂₀aryl aralkyl, akylcarbonyloxy, arylcarbonyloxy, alkyloxy, alklylthio, alkylamino, alkyloxyalkyl, alkanoyl, alkylcarbonylalkyl, optionally substituted by one or more substituents independently selected from the group comprising alkyl, aralkyl, aryl, cycloalkyl, alkyloxycarbonyl, carboxyl, aminocarbonyl, hydroxy, cyano, halogen or amino optionally mono- or disubstituted wherein the substituents are independently selected from the group comprising alkyl, aryl, aralkyl, aryloxy, arylamino, aryloxyalkyl, arylaminoalkyl, aralkoxy, alkoxy.

In further aspects, the present invention relates to pharmaceutical or phytopharmaceutical composition comprising said compounds of formula (1) and to therapeutic uses thereof. The present invention will be further disclosed in detail hereunder. Examples are given which will further support the description.

### DETAILED DESCRIPTION

The present invention provided inventive compounds of formula (1) and salts, stereoisomeric forms, racemic mixtures, prodrugs, esters and metabolites thereof, wherein 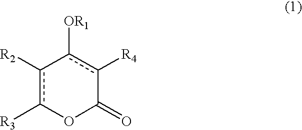
R₁ is a glycosyl moiety, hydroxyl-protected acetate derivatives thereof or amino derivatives thereof;
R₂, R₃ an R₄ are, each independently, selected from the group comprising hydrogen, C₁₋₆alkyl, C₂₋₂₀alkenyl, C₆₋₂₀arylalkyl, C₃₋₇cycloalkyl, C₃₋₇cycloalkylC₁₋₆alkyl, C₆₋₂₀aryl aralkyl, akylcarbonyloxy, arylcarbonyloxy, alkyloxy, alklylthio, alkylamino, alkyloxyalkyl, alkanoyl, alkylcarbonylalkyl, optionally substituted by one or more substituents independently selected from the group comprising alkyl, aralkyl, aryl, cycloalkyl, alkyloxycarbonyl, carboxyl, aminocarbonyl, hydroxy, cyano, halogen or amino optionally mono- or disubstituted wherein the substituents are independently selected from the group comprising alkyl, aryl, aralkyl, aryloxy, arylamino, aryloxyalkyl, arylaminoalkyl, aralkoxy, alkoxy.

As used herein the term “glycosylated” or “glycosyl” moiety refers to a saccharyl moiety such as a mono-, di-, or an oligo-saccharide moiety, a hydroxy-substituted cyclohexyl moiety, the amino derivatives thereof or the hydroxyl-protected acetate derivatives thereof. The term “saccharyl” as used herein refers to saccharide moeity. Exemplary monosaccharide moiety includes but is not limited to a pentosyl, an hexosyl, or a heptosyl moiety. The glycosyl moiety R₁ may also be substituted with various groups. Such substitutions may include lower alkyl, lower alkoxy, acyl, carboxy, carboxyamino, amino, acetamido, halo, thio, nitro, keto, and phosphatyl groups, wherein the substitution may be at one or more positions on the saccharide. Moreover, the glycosyl may also be present as a deoxy glycosyl. The hydroxy-substituted cyclohexyl moiety includes but is not limited to mono-hydroxycyclohexyl group such as 2-, 3- or 4-hydroxycyclohexyl group, a di-hydroxycyclohexyl group such as 2,3-, 2,4-, 2,5-, 2,6-, 3,4-, or 3,5-dihydroxycyclohexyl) group, a tri-hydroxycyclohexyl group such as 2,3,4-, 2,3,5-, 2,3,6-, 3,4,5-, or 3,4,6-trihydroxycyclohexyl group or a tetra-hydroxycyclohexyl group such as 2,3,4,5-, 2,3,4,6-, or 2,3,5,6-tetrahydroxycyclohexyl group, hydroxyl-protected acetate derivatives thereof or amino derivatives thereof.

In an embodiment of the present invention, said glycosyl moiety is a saccharyl moiety, a hydroxy-substituted cyclohexyl moiety, hydroxyl-protected acetate derivatives thereof or amino derivatives thereof.

In another preferred embodiment of the present invention, R₁ is selected from the group comprising glucopyranosyl, fructosyl, galactopyranosyl, mannopyranosyl, ribosyl, ribulosyl, xylulosyl, erythrosyl, threosyl, sorbosyl, psicosyl, tagatosyl, fucosyl, arabinosyl, xylofuranosyl, lyxosyl, talosyl, idosyl, gulosyl, altrosyl, allosyl, mannoheptulosyl, sedoheptulosyl, maltosyl, lactosyl, glucofuranosyl, sucrosyl, cellobiosyl, trehalosyl, gentiobiosyl, melibiosyl, turanosyl, sophorosyl, isosucrosyl, raffinosyl, gentianosyl, 2-amino-2-deoxy glucosyl, 2-amino-2-deoxy galactosyl, 2-amino-1,3-cyclohexanediol, hydroxyl-protected acetate derivatives thereof or amino derivatives thereof.

In yet another embodiment of the present invention, R₂, R₃ an R₄ are, each independently, selected from the group comprising hydrogen, C₁₋₆alkyl, C₂₋₂₀alkenyl, C₆₋₂₀arylalkyl, C₃₋₇cycloalkyl, C₃₋₇cycloalkylC₁₋₆alkyl, C₆₋₂₀aryl and R₁ is selected from the group comprising glucopyranosyl, fructosyl, galactopyranosyl, mannopyranosyl, ribosyl, ribulosyl, xylulosyl, erythrosyl, threosyl, sorbosyl, psicosyl, tagatosyl, fucosyl, arabinosyl, xylofuranosyl, lyxosyl, talosyl, idosyl, gulosyl, altrosyl, allosyl, mannoheptulosyl, sedoheptulosyl, maltosyl, lactosyl, glucofuranosyl, sucrosyl, cellobiosyl, trehalosyl, gentiobiosyl, melibiosyl, turanosyl, sophorosyl, isosucrosyl, raffinosyl, gentianosyl, 2-amino-2-deoxy glucosyl, 2-amino-2-deoxy galactosyl, 2-amino-1,3-cyclohexanediol, hydroxyl-protected acetate derivatives thereof or amino derivatives thereof.

The present invention provides inventive compounds of formula (1) having more particularly the formula (2), wherein R₁, R₂, R₃ and R₄ have the same meaning as that defined above.

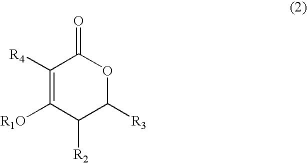

In another embodiment, the present invention provides compounds having the formula (3), wherein R₁, R₂, R₃ and R₄ have the same meaning as that defined above.

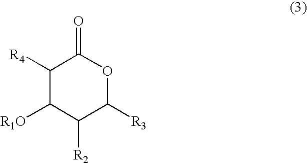

In yet another embodiment, the present invention provides compounds having the formula (4), wherein R₁, R₂, R₃ and R₄ have the same meaning as that defined above.

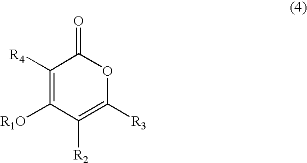

More in particular, the present invention provides compounds of formula (1), (2), (3) or (4) wherein R₁ is glucopyranosyl or galactopyranosyl, R₂, R₃ and R₄ are each independently C₁₋₆alkyl.

In another embodiment, the present invention relates to compounds having the formula (5), 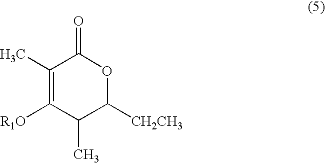
wherein R₁ is selected from the group comprising glucopyranosyl, fructosyl, galactopyranosyl, mannopyranosyl, ribosyl, ribulosyl, xylulosyl, erythrosyl, threosyl, sorbosyl, psicosyl, tagatosyl, fucosyl, arabinosyl, xylofuranosyl, lyxosyl, talosyl, idosyl, gulosyl, altrosyl, allosyl, mannoheptulosyl, sedoheptulosyl, maltosyl, lactosyl, glucofuranosyl, sucrosyl, cellobiosyl, trehalosyl, gentiobiosyl, melibiosyl, turanosyl, sophorosyl, isosucrosyl, raffinosyl, gentianosyl, 2-amino-2-deoxy glucosyl, 2-amino-2-deoxy galactosyl, 2-amino-1,3-cyclohexanediol, hydroxyl-protected acetate derivatives thereof or amino derivatives thereof.

In another embodiment, the present invention relates to compounds having the formula (6), 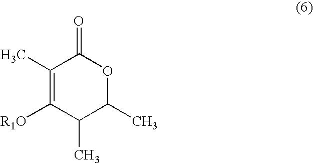
wherein R₁ is selected from the group comprising glucopyranosyl, fructosyl, galactopyranosyl, mannopyranosyl, ribosyl, ribulosyl, xylulosyl, erythrosyl, threosyl, sorbosyl, psicosyl, tagatosyl, fucosyl, arabinosyl, xylofuranosyl, lyxosyl, talosyl, idosyl, gulosyl, altrosyl, allosyl, mannoheptulosyl, sedoheptulosyl, maltosyl, lactosyl, glucofuranosyl, sucrosyl, cellobiosyl, trehalosyl, gentiobiosyl, melibiosyl, turanosyl, sophorosyl, isosucrosyl, raffinosyl, gentianosyl, 2-amino-2-deoxy glucosyl, 2-amino-2-deoxy galactosyl, 2-amino-1,3-cyclohexanediol, hydroxyl-protected acetate derivatives thereof or amino derivatives thereof.

In another embodiment, the present invention relates to compounds having the formula (7), 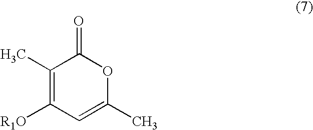
wherein R₁ is selected from the group comprising glucopyranosyl, fructosyl, galactopyranosyl, mannopyranosyl, ribosyl, ribulosyl, xylulosyl, erythrosyl, threosyl, sorbosyl, psicosyl, tagatosyl, fucosyl, arabinosyl, xylofuranosyl, lyxosyl, talosyl, idosyl, gulosyl, altrosyl, allosyl, mannoheptulosyl, sedoheptulosyl, maltosyl, lactosyl, glucofuranosyl, sucrosyl, cellobiosyl, trehalosyl, gentiobiosyl, melibiosyl, turanosyl, sophorosyl, isosucrosyl, raffinosyl, gentianosyl, 2-amino-2-deoxy glucosyl, 2-amino-2-deoxy galactosyl, 2-amino-1,3-cyclohexanediol, hydroxyl-protected acetate derivatives thereof or amino derivatives thereof.

In a preferred embodiment, R₁ in said compounds of formula (5), (6) and (7) is β-D-glucopyranosyl.

The present invention provides in particular, compound having the formula (8), (9) or (10) or stereoisomers thereof.

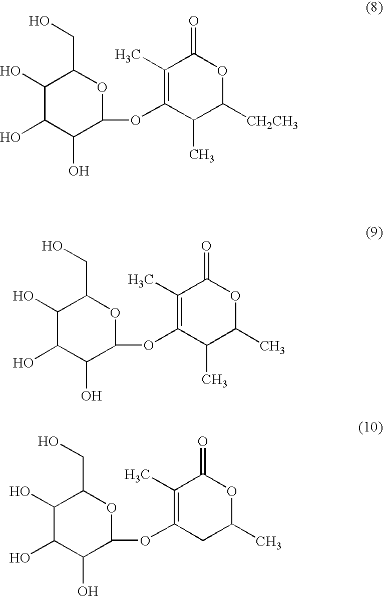

For example, the present invention provides the compounds having the formula (11), (12) or (13) respectively named herein cornicinine, norcornicinine and X-cornicinine.

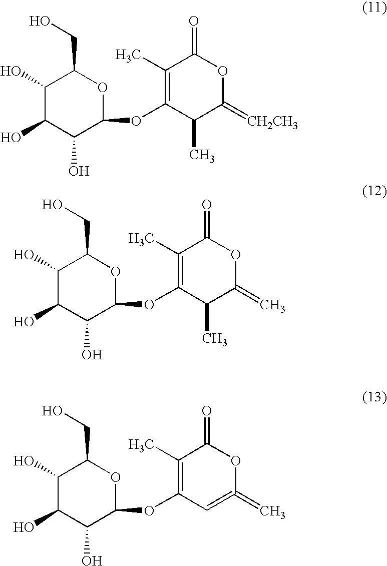

It was surprisingly found that the glycosylated δ-lactone of trans configuration, examples of which are illustrated under formula (11) or (12) have a biological activity.

The articles “a” and “an” are used herein to refer to one or to more than one, i.e. to at least one, the grammatical object of the article. By way of example, “a compound” means one compound or more than one compound.

The term “C₁₋₆alkyl” as a group or part of a group defines straight and branched chained saturated hydrocarbon radicals having from 1 to 6 carbon atoms such as, for example, methyl, ethyl, propyl, butyl and 2-methyl-propyl, pentyl, hexyl, 2-methylbutyl, 3-methylpentyl and the like.

The term “C₂₋₂₀alkenyl” as a group or part of a group defines straight and branched chained hydrocarbon radicals having from 2 to 20 carbon atoms containing at least one double bond such as, for example, ethenyl, propenyl, butenyl, pentenyl, hexenyl and the like.

The term “C₃₋₇cycloalkyl” as a group or part of a group is generic to cyclopropyl, cyclobutyl, cyclopentyl, cyclohexyl or cycloheptyl.

The term “aryl” as a group or part of a group is meant to include phenyl and naphtyl which both may be optionally substituted with one or more substituents independently selected from C₁₋₆alkyl, C₁₋₆alkyloxy, halogen, hydroxy, optionally mono- or disubstituted amino, nitro, cyano, haloC₁₋₆alkyl, carboxyl, C₁₋₆alkoxycarbonyl, C₃₋₇cycloalkyl, heterocycle, optionally mono- or disubstituted aminocarbonyl, optionally mono- or disubstituted aminoC₁₋₆alkyl, methylthio, methylsulfonyl, and phenyl optionally substituted with one or more substituents selected from C₁₋₆alkyl, C₁₋₆alkyloxy, halogen, hydroxy, optionally mono- or disubstituted amino, nitro, cyano, haloC₁₋₆alkyl, carboxyl, C₁₋₆alkoxycarbonyl, C₃₋₇cycloalkyl, optionally mono- or disubstituted aminocarbonyl, methylthio and methylsulfonyl; whereby the optional substituents on any amino function are independently selected from C₁₋₆alkyl, C₁₋₆alkylcarbonyl.

As used herein, the term “halide” or “halo” as a group or part of a group is generic for fluoro, chloro, bromo or iodo.

All stereoisomers of the inventive compounds are included within the scope of the invention, as pure compounds as well as mixtures thereof. Individual enantiomers, diastereomers, geometric isomers, and combinations and mixtures thereof are all encompassed by the present invention.

Pure stereoisomeric forms of the compounds and intermediates as mentioned herein are defined as isomers substantially free of other enantiomeric or diastereomeric forms of the same basic molecular structure of said compounds or intermediates. In particular, the term ‘stereoisomerically pure’ concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms ‘enantiomerically pure’ and ‘diastereomerically pure’ should be understood in a similar way, but then having regard to the enantiomeric excess, respectively the diastereomeric excess of the mixture in question.

Pure stereoisomeric forms of the compounds and intermediates of this invention may be obtained by the application of art-known procedures. For instance, enantiomers may be separated from each other by the selective crystallization of their diastereomeric salts with optically active acids. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound will be synthesized by stereospecific methods of preparation. These methods will advantageously employ enantiomerically pure starting materials.

The diastereomeric racemates of formula (1) can be obtained separately by conventional methods. Appropriate physical separation methods which may advantageously be employed are, for example, selective crystallization and chromatography, e.g. column chromatography.

The absolute configuration of each asymmetric center that may be present in the compounds of formula (1) may be indicated by the stereochemical descriptors R and S, this R and S notation corresponding to the rules described in Pure Appl. Chem. 1976, 45, 11 30.

Furthermore, some of the crystalline forms for the compounds may exist as polymorphs and as such are included in the present invention. In addition, some of the compounds may form solvates with water (i.e., hydrates) or common organic solvents, and such solvates are also encompassed within the scope of this invention.

Protected forms of the inventive compounds are included within the scope of the present invention. A variety of protecting groups are disclosed, for example, in T. H. Greene and P. G. M. Wuts, Protective Groups in Organic Synthesis, Third Edition, John Wiley & Sons, New York (1999), which is incorporated herein by reference in its entirety. For example, hydroxy protected forms of the inventive compounds are those where at least one of the hydroxyl groups is protected by a hydroxy protecting group. Illustrative hydroxyl protecting groups include but not limited to tetrahydropyranyl; benzyl; methylthiomethyl; ethylthiomethyl; pivaloyl; phenylsulfonyl; triphenylmethyl; trisubstituted silyl such as trimethyl silyl, triethylsilyl, tributylsilyl, tri-isopropylsilyl, t-butyldimethylsilyl, tri-t-butylsilyl, methyldiphenylsilyl, ethyldiphenylsilyl, t-butyldiphenylsilyl and the like; acyl and aroyl such as acetyl, pivaloylbenzoyl, 4-methoxybenzoyl, 4-nitrobenzoyl and aliphatic acylaryl and the like. Keto groups in the inventive compounds may similarly be protected.

The present invention includes within its scope prodrugs of the compounds of this invention. In general, such prodrugs are functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to a subject in need thereof. Conventional procedures for the selection and preparation of suitable prodrug derivatives are described, for example, in “Design of Prodrugs”, H. Bundgaard ed., Elsevier, 1985.

The present invention is also intended to include all isotopes of atoms occurring on the present compounds. Isotopes include those atoms having the same atomic number but different mass numbers. By way of general example and without limitation, isotopes of hydrogen include tritium and deuterium. Isotopes of carbon include ¹³C and ¹⁴C.

The present invention therefore provides compounds of formula (1) which can be used as phytopharmaceutic and pharmaceutic agents in the treatment of plants and animals against microbial infections including bacterial, yeast, fungal, viral and protozoan infections.

The present invention further relates to a pharmaceutical or a phytopharmaceutical composition comprising at least one compound according the present invention and a pharmaceutically acceptable carrier. Said compound is preferably provided in a therapeutically effective amount. Pharmaceutical acceptable salts or ester of said compounds can also be used in said composition.

The term “therapeutically effective amount” as used herein, means that amount of active compound or pharmaceutical agent that elicits the biological or medicinal response in a tissue system, plant, animal or human that is being sought by a researcher, veterinarian, medical doctor or other clinician, which includes alleviation of the symptoms of the disease or disorder being treated.

The term “composition” is intended to encompass a product comprising the specified ingredients in the specified amounts, as well as any product that results, directly or indirectly, from combinations of the specified ingredients in the specified amounts.

The term “pharmaceutically acceptable salt” is a salt of one or more of the inventive compounds. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like.

The term “pharmaceutically acceptable ester” is an ester that hydrolyzes in vivo into a compound of the present invention or a salt thereof. Illustrative examples of suitable ester groups include, for example, those derived from pharmaceutically acceptable aliphatic carboxylic acids such as formates, acetates, propionates, butyrates, acrylates, and ethylsuccinates.

In one embodiment, the inventive compounds and compositions are used as a medicament. In another embodiment, the inventive compounds and compositions are used as immunosuppressive agents. In another embodiment, the inventive compounds and compositions are used as neurotrophic agents. In yet another embodiment, the inventive compounds and composition are used as agents to treat inflammatory disorders, particularly inflammatory skin diseases such as psoriasis and dermatitis.

In a further embodiment, the inventive compounds and compositions are used as a biostatic. In another embodiment, the inventive compounds and compositions are used as biocides. In another embodiment, the inventive compounds and compositions are used as antibiotic.

In yet other embodiments, the inventive compounds and compositions are used as an antifungal. In another embodiment, the inventive compounds and compositions are used as cholesterol lowering agents. In yet another embodiment, the inventive compounds and composition are used as drugs to treat cancer.

The method generally comprises administering a therapeutically effective amount of an inventive compound to a subject in need thereof. The present compounds can thus be used in plants, insects, animals, preferably in mammals, and in particular in humans as pharmaceuticals per se, in mixtures with one another or in the form of pharmaceutical preparations.

The compounds of the present invention can be administered on an as-need basis and may be given to patients continuously or an intermittent basis, such as hourly, semi-daily, daily, semi-weekly, weekly, semi-monthly, or monthly intervals. In general, the dosage is the minimum amount of compound that is needed to effectuate the desired effect.

Furthermore, the present invention relates to pharmaceutical preparations which as active constituents contain an effective dose of at least one of the compounds of formula (1) in addition to customary pharmaceutically acceptable carriers and auxiliaries. The pharmaceutical preparations normally contain 0.1 to 90% by weight of a compound of formula (1). The pharmaceutical preparations can be prepared in a manner known per se to one of skill in the art. For this purpose, at least one of a compound of formula (1), together with one or more solid or liquid pharmaceutically acceptable carrier and/or auxiliaries and, if desired, in combination with other pharmaceutical active compounds, are brought into a suitable administration form or dosage form which can then be used as a pharmaceutical in human medicine or veterinary medicine.

Pharmaceuticals or medicaments which contain a compound according to the invention can be administered orally, parenterally, e.g., intravenously, rectally, by inhalation, or topically, the preferred administration being dependent on the individual case, e.g., the particular course of the disorder to be treated.

The term “pharmaceutically acceptable carrier” is a medium that is used to prepare a desired dosage form of the inventive compound. A pharmaceutically acceptable carrier includes solvents, diluents, or other liquid vehicle; dispersion or suspension aids; surface active agents; isotonic agents; thickening or emulsifying agents, preservatives; solid binders; lubricants and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe, ed. (Amer. Pharmaceutical Assoc. 2000), both of which are incorporated herein by reference in their entireties, disclose various carriers used in formulating pharmaceutical compositions and known techniques for the preparation thereof.

The person skilled in the art is familiar on the basis of his expert knowledge with the auxiliaries which are suitable for the desired pharmaceutical formulation. Beside solvents, gel-forming agents, suppository bases, tablet auxiliaries and other active compound carriers, antioxidants, dispersants, emulsifiers, antifoams, flavor corrigents, preservatives, solubilizers, agents for achieving a depot effect, buffer substances or colorants are also useful.

For an oral administration form, compounds of the present invention are mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms.

For subcutaneous or intravenous administration, the active compounds, if desired with the substances customary therefor such as solubilizers, emulsifiers or further auxiliaries, are brought into solution, suspension, or emulsion. The compounds of formula (1) can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned.

Suitable pharmaceutical of phytopharmaceutical formulations for administration in the form of aerosols or sprays are, for example, solutions, suspensions or emulsions of the compounds of formula (I) or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Such a preparation customarily contains the active compound in a concentration from approximately 0.1 to 50%, in particular from approximately 0.3 to 3% by weight.

In order to enhance the solubility and/or the stability of the compounds of formula (I) in pharmaceutical compositions, it can be advantageous to employ α-, β- or γ-cyclodextrins or their derivatives. Also co-solvents such as alcohols may improve the solubility and/or the stability of the compounds of formula (I) in pharmaceutical compositions. In the preparation of aqueous compositions, addition salts of the subject compounds are obviously more suitable due to their increased water solubility.

Appropriate cyclodextrins are α, β- or γ-cyclodextrins (CDs) or ethers and mixed ethers thereof wherein one or more of the hydroxy groups of the anhydroglucose units of the cyclodextrin are substituted with C₁₋₆alkyl, particularly methyl, ethyl or isopropyl, e.g. randomly methylated β-CD; hydroxyC₁₋₆alkyl, particularly hydroxyethyl, hydroxypropyl or hydroxybutyl; carboxyC₁₋₆alkyl, particularly carboxymethyl or carboxyethyl; C₁₋₆alkylcarbonyl, particularly acetyl; C₁₋₆alkyloxycarbonylC₁₋₆alkyl or carboxyC₁₋₆alkyloxyC₁₋₆alkyl, particularly carboxymethoxypropyl or carboxyethoxypropyl; C₁₋₆alkylcarbonyloxyC₁₋₆alkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxypropyl-γ-CD and (2-carboxymethoxy)propyl-β-CD, and in particular 2-hydroxypropyl-β-CD (2-HP-β-CD).

The term mixed ether denotes cyclodextrin derivatives wherein at least two cyclodextrin hydroxy groups are etherified with different groups such as, for example, hydroxyl-propyl and hydroxyethyl.

An interesting way of formulating the present compounds in combination with a cyclodextrin or a derivative thereof has been described in EP-A-721,331. Although the formulations described therein are with antifungal active ingredients, they are equally interesting for formulating the compounds of the present invention. The formulations described therein are particularly suitable for oral administration and comprise an antifungal as active ingredient, a sufficient amount of a cyclodextrin or a derivative thereof as a solubilizer, an aqueous acidic medium as bulk liquid carrier and an alcoholic co-solvent that greatly simplifies the preparation of the composition. Said formulations may also be rendered more palatable by adding pharmaceutically acceptable sweeteners and/or flavors.

Other convenient ways to enhance the solubility of the compounds of the present invention in pharmaceutical compositions are described in WO-94/05263, PCT application No. PCT/EP98/01773, EP-A-499,299 and WO 97/44014, all incorporated herein by reference.

More in particular, the present compounds may be formulated in a pharmaceutical composition comprising a therapeutically effective amount of particles consisting of a solid dispersion comprising (a) a compound of formula (1), and (b) one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered.

The term “a solid dispersion” also comprises dispersions which are less homogenous throughout than solid solutions. Such dispersions are not chemically and physically uniform throughout or comprise more than one phase.

The water-soluble polymer in the particles is conveniently a polymer that has an apparent viscosity of 1 to 100 mPa.s when dissolved in a 2% aqueous solution at 20° C. solution.

Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 are generally water soluble. Methoxy degree of substitution refers to the average number of methyl ether groups present per anhydroglucose unit of the cellulose molecule. Hydroxy-propyl molar substitution refers to the average number of moles of propylene oxide which have reacted with each anhydroglucose unit of the cellulose molecule.

The particles as defined hereinabove can be prepared by first preparing a solid dispersion of the components, and then optionally grinding or milling that dispersion. Various techniques exist for preparing solid dispersions including melt-extrusion, spray-drying and solution-evaporation.

It may further be convenient to formulate the present compounds in the form of nanoparticles which have a surface modifier adsorbed on the surface thereof in an amount sufficient to maintain an effective average particle size of less than 1000 nm. Useful surface modifiers are believed to include those which physically adhere to the surface of the antiretroviral agent but do not chemically bind to the antiretroviral agent.

Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

Yet another interesting way of formulating the present compounds involves a pharmaceutical composition whereby the present compounds are incorporated in hydrophilic polymers and applying this mixture as a coat film over many small beads, thus yielding a composition with good bioavailability which can conveniently be manufactured and which is suitable for preparing pharmaceutical dosage forms for oral administration.

Said beads comprise (a) a central, rounded or spherical core, (b) a coating film of a hydrophilic polymer and an antiretroviral agent and (c) a seal-coating polymer layer.

Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof.

The dose of the present compounds or of the physiologically tolerable salt(s) thereof to be administered depends on the individual case and, as customary, is to be adapted to the conditions of the individual case for an optimum effect. Thus it depends, of course, on the frequency of administration and on the potency and duration of action of the compounds employed in each case for therapy or prophylaxis, but also on the nature and severity of the infection and symptoms, and on the sex, age, weight and individual responsiveness of the human or animal to be treated and on whether the therapy is acute or prophylactic. Customarily, the daily dose of a compound of formula (I) in the case of administration to a patient approximately 75 kg in weight is 1 mg to 1 g, preferably 3 mg to 0.5 g. The dose can be administered in the form of an individual dose, or divided into several, e.g. two, three, or four, individual doses.

The new compounds or pharmaceutical compositions thereof are useful as medicament and more particularly as medicament for the treatment of cancer and drug resistant cancer. Said new compounds are therefore useful as antitumor agent, and may be used for the preparation of medicament for treating cancer. The present invention furthermore relates to a method of treating a patient suffering from cancer, wherein a compound as described above is administered to the patient.

The inventive compound according to the invention can also be used in the preparation of a medicament for the treatment of fungal diseases. In another embodiment, said compounds can be used in the preparation of a medicament for the treatment of cholesterol induced diseases. The inventive compound according to the invention can be also be used in the preparation of a medicament for the treatment of immune diseases.

The present invention also provides a method for treating or preventing diseases caused by microorganisms, which comprises administering said compound to human being, plants or animals.

In another aspect of the present invention, the inventive compounds as described herein can be used as intermediates in the synthesis of other compounds non limiting examples of which include polyketides. These intermediates can then be incorporated into pathways for the synthesis of novel polyketides using native or modified polyketide synthase (PKS) systems or conventional chemical synthesis.

The present invention also provides for a method for the preparation of a polyketide, wherein said method comprises treating a polyketide synthase (PKS) enzyme complex with the compound according to the invention under conditions wherein said polyketide is formed. In an embodiment of said method, said PKS is contained in a cell.

The polyketides prepared encompassed in the present invention the large family of diverse compounds ultimately synthesized from 3-carbon units through a series of Claisen-type condensations and subsequent modifications. Members of this group include antibiotics such as tetracyclines, anticancer agents such as daunomycin, and immunosuppressants such as FK506 and rapamycin. The polyketides can be synthesized by polyketide synthases (PKS). As used herein, “polyketide” refers to the immediate product of a polyketide synthase enzyme system. It is generally a lactone of 13-15 C. “Tailored polyketides” refers to the products of subsequent derivatization of the resultant polyketide which occurs through enzymatic treatment by enzymes endogenous to organisms which synthesize polyketide antibiotics. Such tailoring enzymes may add hydroxyl groups, remove hydroxyl or oxo groups, add sugars, modify sugars that have been coupled to the polyketide, and the like. “Derivatized polyketides” refers to polyketides or tailored polyketides which have been modified chemically in ways generally unavailable from straightforward enzymatic treatment. The compounds of the present invention can be provided to a host cell that expresses a PKS but not post PKS modification enzymes (such as hydroxylases and glycosyltransferases) or can be provided to a host cell that expresses both types of enzymes.

Recombinant host cells containing cloned PKS expression vectors can be constructed to express all of the biosynthetic genes for a modified polyketide or only a subset of the same. If only the genes for the PKS are expressed in a host cell that otherwise does not produce polyketide modifying enzymes that can act on the polyketide produced, then the host cell produces unmodified polyketides. Such unmodified polyketides can be hydroxylated and glycosylated, for example, by adding them to the culture medium of a strain such as, for example, Streptomyces antibioticus or Saccharopolyspora erythraea, that contains the requisite modification enzymes. The resulting polyketides can further be modified by chemical means or by feeding to a native antibiotic producing host for glycosylation or further modification.

The compounds of formula (1) can be produced not only by chemical synthesis or biosynthesis but also from extracts of insects containing the compound. As an organism containing the compound, Nephrotoma cornicina may be exemplarily presented.

The compounds of formula (1) can be utilized as an antifoulant by preparing coatings, solutions, emulsions, etc., each using the compound. When used as a coating, the compound is blended with a coating composition so as to prepare an antifouling coating to be applied to the bottoms of ships, underwater structure, water channels for cooling water, etc.

Particular reaction procedures to make the present compounds of formula (1) are described below. In the preparations described below, the reaction products may be isolated from the medium and, if necessary, further purified according to methodologies generally known in the art such as, for example, extraction, crystallization, trituration and chromatography.

The compounds of formula (1) may be isolated from Tipulidae species such as Nephrotoma cornicina.

The compound of formula (1) may also be prepared using combinatorial biosynthesis. For example, a compound of formula (1) may be prepared by using small hybrid PKS systems, followed by a glycosylation step.

The compounds of formula (1a) may also be synthetically prepared, as described in the synthetic scheme 1, wherein (i) is an acylation procedure using n-BuLi and BrCH(R₄)COBr, (ii) Sml₂, −78° C., then intermediate (a), (iii) 1) H₂O₂, LiOH, THF 2) HCl, THF, 40° C. (iv) glycosylation step. The stereochemistry can be controlled by using the desired chiral oxazolidinone.

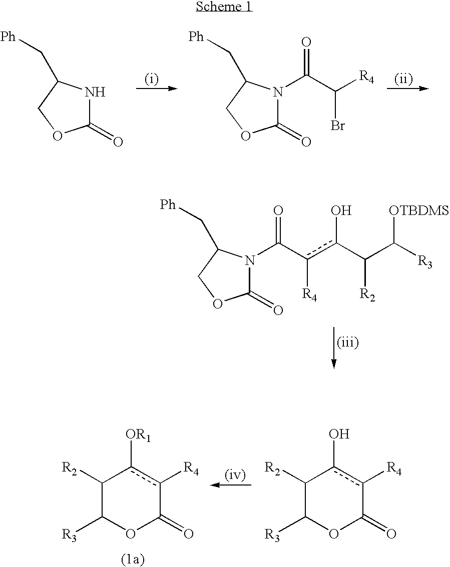

Intermediates (a) may be prepared by adapting the procedure as described in Wilkinson et al. Tet. Lett. 1998, 39, 9827, as illustrated under scheme (2), wherein (i′) 1) Bu₂BOTf, Et₃N 2) R₃CHO, (ii′) 1) TBDMSCI, imidazole, DMF 2) LiBH₄, H2O, (iii′) (COCl)₂, DMSO, Et₃N. The desired stereochemistry can be obtained by using different isomers of the propionate oxazolidinone chiral auxiliary as starting material.

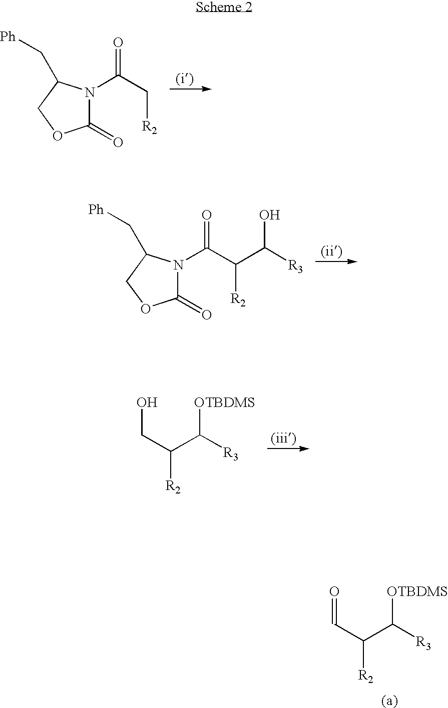

Compounds of formula (2) can be prepared according to the synthetic scheme 3 or 4. The synthetic route of scheme 3 comprises treating an acyl halide of the formula (II) with a ketone acetal of the formula (III) to produce a δ-hydroxy-protected-β-enol ether ester of the formula (IV), followed by removing at least one of the protecting groups R₆ and R₈, and contacting the product with an acid to produce the δ-lactone of formula (V), which is then glycosylated to give compound of formula (2), wherein R₁, R₂, R₃ an R₄ have the same meaning as that defined above, X is a halide, R₆ is an OH protecting group, and R₇ and R₅ are, each independently, C₁₋₆alkyl, C₆₋₂₀arylalkyl or SiR₁₁R₁₂R₁₃, wherein R₁₁, R₁₂, R₁₃ are C₁₋₆alkyl or phenyl, R₈ is H or R₇.

Said process includes treating an acyl halide of the formula (II) with a ketene acetal of the formula (III) under conditions sufficient to produce a δ-hydroxy-protected-β-enol ether ester of the formula (IV). A variety of protecting groups, including protecting groups for hydroxy and carboxylic acid functional groups, are known in the art, and can be employed. Examples of many of the possible protecting groups can be found in Protective Groups in Organic Synthesis, 3rd edition, T. W. Greene and P. G. M. Wuts, John Wiley & Sons, New York, 1999, which is incorporated herein by reference in its entirety.

The reaction is typically carried out in a conventional aprotic organic solvent, such as THF, toluene, heptane, hexane or mixtures thereof, in the presence of a tertiary amine including trialkyl amine, such as triethyl amine or tributyl amine, under preferably an inert atmosphere described above.

The crude δ-hydroxy-protected-β-enol ether ester (IV) can be purified, e.g., by distillation under a reduced pressure or by chromatography, or it can be used directly in the next step without any purification. As used herein, a “crude” compound refers to a compound which is not subject to a separate purification step other then a conventional work-up of the reaction.

Processes of the present invention also include treating δ-hydroxy-protected-β-enol ether ester (IV) under reaction conditions sufficient to remove at least one of the protecting groups (i.e., R₆ and/or R₈, preferably at least R₈) and contacting the resulting deprotected compound with an acid to produce the δ-lactone (V).

A particularly useful ketene acetal (III) is a silyl ketene acetal in which R₇ is a moiety of the formula —SiR₁₁R₁₂R₁₃ and R₅ is C₁₋₆alkyl, C₅₋₂₀aryl or C₆₋₂₀arylalkyl. Silyl ketenes can be prepared by any of the currently known methods. Some of the methods for preparing silyl ketenes are disclosed in Miura et al., Bull. Chem. Soc. Jpn., 1991, 64, 1542-1553; Umemoto and Gotoh, Bull. Chem. Soc. Jpn., 1987, 60, 3823-3825; Sugimoto et al., Chem. Lett., 1991, 1319-1322; Miura et al., Bull. Chem. Soc. Jpn., 1992, 65, 1513-1521; and Shono et al., J. Org. Chem., 1984, 49, 1056-1059, which are incorporated herein by reference in their entirety.

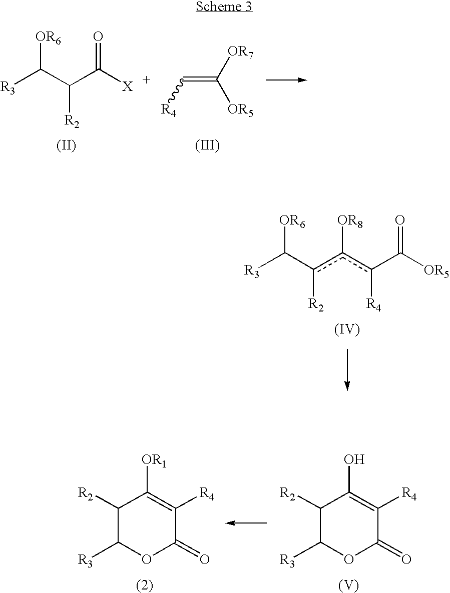

The synthetic route of scheme 4 comprises treating an acyl halide of the formula (II) with a malonate half acid of formula (VI) to produce a δ-hydroxy-β-ketoester of the formula (VII), followed by treating said compound with an acid to produce the δ-lactone of formula (V), which is then glycosylated to produce compound of formula (2), wherein R₁, R₂, R₃ an R₄ have the same meaning as that defined above, and R₆ is an OH protecting group, R₁₀ is C₁₋₆alkyl, C₆₋₂₀aryl, C₆₋₂₀arylalkyl, X is a halide such as a chloride and R₉ is H or an OH protecting group.

The reaction between the acyl halide (II) and the malonate half acid (VI) can be carried out in the presence of a metal salt and a tertiary amine base. See for example, Rathke and Cowan, J. Org. Chem., 1985, 50, 2622-2624, and Clay et al., Synthesis, 1993, 290-292, which are incorporated herein by reference in their entirety. The reaction can be conducted under an aprotic organic solvent, such as n-butyl ether, THF, acetonitrile, methylene chloride, dimethoxyethane (DME), methyl t-butyl ether (MTBE), toluene, 2-methyl tetrahydrofuran (2-Me-THF), with THF being the preferred solvent, and followed by a basic hydrolysis to decarboxylate compound of the formula (VIII).

Exemplary metal salts include magnesium salts such as magnesium halides (MgCl₂, MgBr₂ and Mgl₂); manganese salts, (manganese halides and manganese acetates); lithium salts (lithium halides); samarium salts (samarium halides); and mixtures of sodium and lithium salts (mixtures of sodium halides and lithium halides). Preferably, the metal salt is a magnesium salt, more preferably magnesium chloride.

Exemplary tertiary amine bases, which are useful in the present invention, include trialkylamines, such as triethylamine, diethylisopropylamine and tributylamine; and other tertiary amines. Preferably, the tertiary amine base is a trialkylamine, more preferably triethylamine, diethylisopropylamine or tributylamine.

The resulting δ-hydroxy-β-ketoester (VII) can be isolated or preferably used directly without isolation and treated with an acid under conditions sufficient to produce the δ-lactone (V).

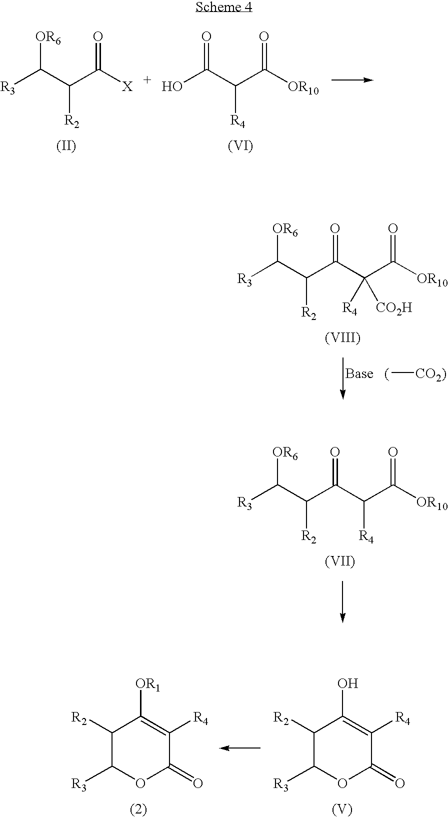

As used herein, the term “treating”, “contacting” or “reacting” refers to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The methods of synthesis described herein are suitable for liquid phase as well as solid-phase combinatorial synthesis. The present invention also encompasses the above mentioned synthesis wherein the radical R₁ is already present in the starting synthon, optionally under a protected form.

To obtain the glycosylated compound according to the invention, an unprotected or suitably protected derivative of the δ-lactone can be reacted under standard glycosidic-bond forming conditions with an activated and/or suitably protected glycosyl derivative.

The glycosyl moiety can be linked to the δ-lactone in a stereospecific manner. Preferably, the glycosyl moiety is linked to the δ-lactone such that a β-glycosidic bond is formed. Protocols useful for this purpose include the Koenigs Knorr procedure (W. Koenigs et al., Ber. Dtsch. Chem. Ges. 34:957 (1901), in which a glycosyl bromide reacts in the presence of a silver catalyst, for example, silver carbonate) and the Helferich procedure (B. Helferich et al., Ber. Dtsch. Chem. Ges. 73:532 (1940) and N. I. Uvarova et al. Carbohydrate Research 83:33-42 (1980)).

The following examples are meant to be illustrative of the present invention. These examples are presented to exemplify the invention and are not to be construed as limiting the invention's scope.

### EXAMPLES

### Example 1

Extract of Nephrotoma cornicina (Linnaeus, 1758) (Tipulidae, Diptera) contains a glycosylated triketide lactone, here named cornicinine. The compound according to the invention is present in the head, thorax, abdomen, legs and wings of the insect, from both sexes. It is active on the seven generally recognised Azolla species and is able to induce the differentiation of all the vegetative cells of A. azollae into akinetes at nanomolar concentrations. It is stable: the activity of N. cornicina extracts is indeed preserved after autoclaving for 30 minutes at 120° C.; moreover, a specimen from the collections of the Royal Belgian Institute of Natural Sciences, collected in 1893, still showed important activity. Dilution bioassays (see methods) showed that 4 μg of dry N. cornicina powder/ml Azolla culture medium induce akinete differentiation.

Approximately 10 000 dried N. cornicina (36 g) were ground, suspended in 750 ml H₂O, treated 60′ in a 100° C. water bath and centrifuged (14 000×g, 60′). The residue was re-suspended in 300 ml H₂O, shacked 15′ and centrifuged (14 000×g, 60′). The two supernatants were grouped, filtered on Macherey-Nagel MN 615 paper and lyophilized. The dry residue (7 g) was suspended in 8 ml H₂O, vortexed and centrifuged (7 800×g, 30′). Six ml of supernatant were filtered on acrodiscs 0.22 μm in four 1.5 ml batches, each filtrate being chromatographed on a 500 ml, 2.5 cm diameter Sephadex G 10 column (eluent: water; 3′/fraction, around 6 ml/fraction) The residue was suspended in 2 ml H₂O, vortexed and centrifuged (7 800×g, 30′). Two ml of supernatant were filtered and chromatographed as above. This last procedure was repeated twice. Absorbance of each fraction was measured at 254 nm and bioassays were performed on 50 μl of each fraction from the seven chromatograms. The most active fractions were grouped and lyophilized.

Gel permeation of crude aqueous extracts from N. cornicina, followed by bioassays and UV/visible spectrometry on the fractions obtained indicated that the active substance has a molecular weight not exceeding a few hundreds g/mol. and a maximum absorbance at 254 nm.

Azolla is a small aquatic fern, which multiplies, most often vegetatively, at the surface of still water bodies, where it can form dense floating carpets. Anabaena azollae is the only known diazotrophic symbiont associated to a pteridophyte, Azolla and the only one linked to a plant throughout its life cycle. Anabaena are filamentous cyanobacteria composed of photosynthesising cells, N₂-fixing cells and sometimes resting, spore-like cells, respectively named vegetative cells, heterocysts and akinetes. A population of A. azollae is permanently present on the stem apex. When a new leaf develops, some Anabaena are engulfed in an invagination of its adaxial epidermis, which progressively forms a central leaf cavity. The imprisoned cyanobacteria multiply and 20-30% of their cells differentiate into heterocysts, whose nitrogenase activity generally covers the ammonium needs of the two symbionts. The cavity sometimes contains a few akinetes.

An Azolla shoot apex +/−3 mm long was excised and placed in a 2 ml Eppendorf tube filled with modified, N-free Hoagland solution to which was added the material to be tested. The culture was performed at 26° C. under constant light intensity of 110 μM. m⁻².sec⁻¹ and with daily adjustment of the liquid level with distilled water to compensate evapotranspiration. Activity was indicated by progressive chlorosis of Azolla, which appears after 5 days. Akinete differentiation can alternatively be followed by observation of leaf squashes under a light microscope, with first symptoms appearing after 48 hours of treatment.

Extract of Nephrotoma cornicina contained glycosylated triketide lactones, some of which are able to induce the differentiation at nanomolar concentrations (5 10⁻⁸ M) of all the vegetative cells of A. azollae into akinetes. This explains the chlorosis of the plants, routinely cultivated on a N-free medium and therefore totally dependent on the nitrogenase activity of the heterocysts, itself dependent on the photosynthetic activity of the vegetative cells.

### Example 2

High performance liquid chromatography (HPLC) of the most active fractions of the extract as performed. An HPLC system of Thermo Separation products (TSP, San José, Calif., USA) consisting of a P1000XR pump and a TSP AS 3000 autosampler was used. Separation was performed on a C18 Novapak column (3.9×300 mm, 4 μm) (Waters, USA), using a linear gradient from 99% water (1% acetonitrile)-1% methanol (maintained for 12 min) to 36% methanol in 35 min at a flow rate of 0.7 ml.min⁻¹. The column was kept at room temperature. Injection volume was 10 μl.

Mass spectra were acquired using a LCQ mass spectrometer equipped with an APCI source (Finnigan MAT, San Jose, Calif., USA). The APCI inlet conditions were: heated vaporization temperature, 450° C.; heated capillary temperature, 170° C.; sheath gas, 40 psi; auxiliary gas, 10 psi; discharge current, 5 μA. Data acquisition and processing were performed with Xcalibur 1.1 software.

A Prep Nova pak C18 column (60° A, 6 μm, 30×300 mm) from Waters was used for preparative separation. Elution was done with a mixture CH₃OH/H₂O (80/20) at a flow rate of 12 ml/min. Samples were filtered on Acrodisc from Eurolab before injection. The injected volume was 100 μl.

HPLC of the most active fractions on a C18 reverse-phase column yielded 35 mg of a pure compound, here named cornicinine, from 36 g of dry N. cornicina.

Atmospheric pressure chemical ionization mass spectrometry (APCI-MS) analysis of cornicinine in positive mode gave a weak pseudo-molecular ion [M+H]⁺ at m/z 333 and a major fragment at m/z 171 due to the loss of a sugar moiety. In negative mode, the major peak corresponds to the chlorinated adduct at 367/369 m/z, a weak pseudo-molecular [M-H]⁻ ion is obtained at 331 m/z and the aglycone fragment ion is detected at m/z=[169].

The aglycone was isolated during a preliminary attempt of purification. HREIMS was performed on this sample, giving a mass of 170.094770 corresponding to C₉H₁₄O₃ (theoretical mass: 170.094294).

NMR experiments were performed on a Bruker Avance 500 MHz. Five mg of the purified extract were dissolved in 0.25 ml of either d6-dmso or D₂O and transferred in a cylindrical insert placed in a 5 mm NMR tube. 1D (¹H, ¹³C, selective INADEQUATE) and 2D (HSQC, HMBC, COSY, ROESY) experiments were recorded using standard procedures implemented on the spectrometer.

Conformational space screening and structure minimization were performed on Silicon Graphics Octane workstation running Sybil software. The AM1 algorithm was selected in all cases.

¹H NMR spectra simulation of minimized 3D structures was performed with ACDLabs HNMR Predictor software (ver 7.0).

The proton spectrum gave evidence for a total of 24 hydrogens distributed in 4 hydroxy groups, 7 in the core of a cyclic sugar ring and 13 in the aglycone substructure. In the latter, we could further identify C_{q}-Me, CH-Me and CH-Et fragments.

An analysis of the sugar signals showed a coupling constant of >7.4 Hz for the anomeric and other ring protons, corresponding to the β form of D-glucopyranose.

The ¹³C spectrum gave evidence for 15 carbon atoms. A 2D heteronuclear single-quantum coherence (HSQC) experiment permits the attribution of the 6 sugar carbons, leaving 9 carbons to be attributed to the aglycone substructure. Subtraction of the 6 carbons from the identified fragments left 3 quaternary atoms (resonance: 169.6, 165.0 and 90.2 ppm).

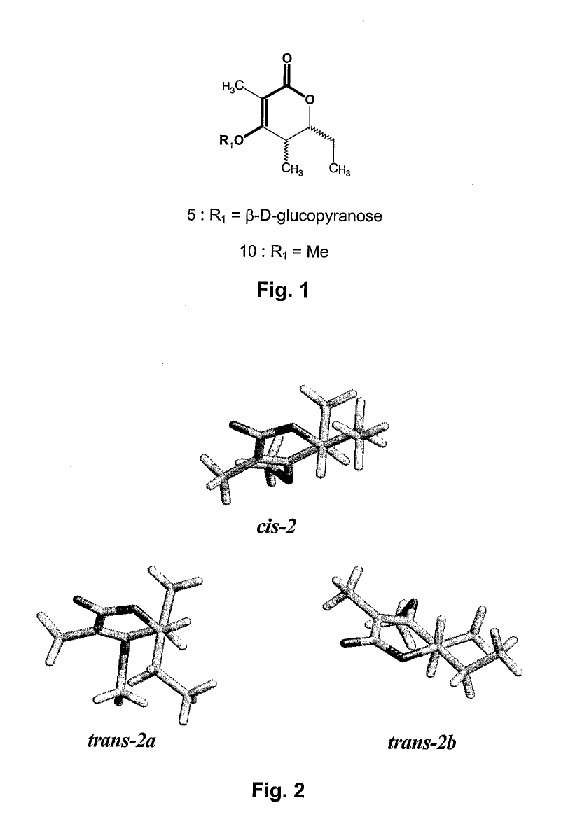

Based on MS analysis, the substructure contains 3 oxygens. Extensive heteronuclear multiple bond correlation (HMBC) and keys selective incredible natural abundance double quantum transition (INADEQUATE) experiments permit to assess the 3 quaternary carbons and the 3 oxygens in the conjugated structure 5 shown in bold in FIG. 1. This type of conjugated structure is further supported by the IR C═O absorption band at 1670 cm⁻¹, which corresponds to a conjugated carbonyl group. 2D's also confirmed the bonding of CH-Me and CH-Et. This bonding was not obvious from the 1D ¹H spectrum since the very small (1.2 Hz) coupling constant between the two CH only appears in DMSO.

The relative stereochemistry of the methyl and ethyl groups in the lactone moiety was studied by nuclear Overhauser enhancements (NOE) measurements and molecular modeling. NOE correlations were found between H₄—H₅ and between the latter and other substituents, including the sugar's anomeric proton. The absence of NOE could not be considered as a structural proof but it has to be noticed that no correlation could be detected between the methyl and ethyl substituent.

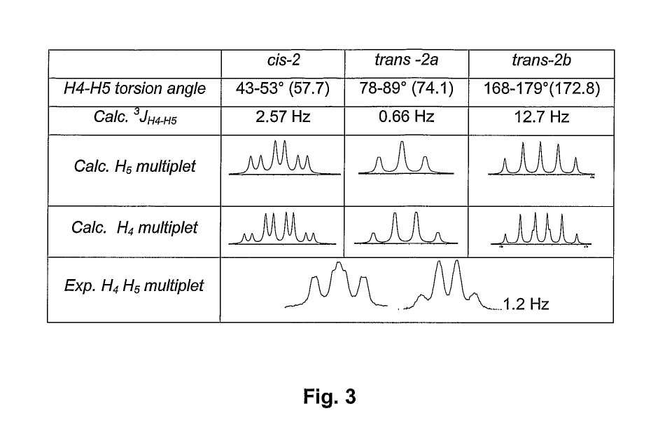

Evaluation of the conformational space and minimization of the various conformers for both cis and trans isomers of 10 gave three stable structures, cis-2, trans-2a and trans-2b (FIG. 2). Simulation of ¹H NMR spectra from 3D structures permits to extract the corresponding H₄—H₅ coupling constants and ¹H NMR multiplet patterns. Results are presented in FIG. 3.

The coupling constant value as well as the multiplet patterns gave evidences for a relative trans-2a stereochemistry between the methyl and ethyl substituents. Moreover, structure trans-2a nicely supports the unobserved NOE in 1; the methyl and ethyl substituents being placed antiperiplanar and distanced by 4.8 A°.

For cornicinine, the NMR spectrometry data obtained are shown in Tables 1 and 2 hereunder.

TABLE 1

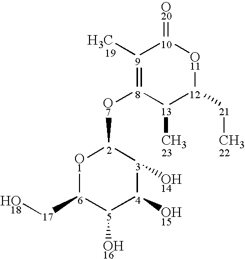

| Carbon (D₂O) | δ (ppm) |
|---|---|
| 19 | 7.95 |
| 22 | 9.32 |
| 23 | 17.24 |
| 21 | 25.61 |
| 13 | 31.33 |
| 17 | 60.01 |
| 5 | 68.98 |
| 4 | 72.53 |
| 6 | 75.20 |
| 3 | 76.05 |
| 12 | 83.82 |
| 2 | 97.23 |
| 9 | 104.57 |
| 8 | 168.90 |
| 10 | 170.13 |

TABLE 2

| Proton (DMSO) | δ (ppm) | J(Hz) |
|---|---|---|
| 22 | 0.87(t) | 3J22-21=7.5 |
| 23 | 1.20(d) | 3J23-13=7.0 |
| 21a | 1.59(m) | |
| 21b | 1.65(m) | |
| 19 | 1.64(s) | |
| 13 | 2.79(q broad) | 3J13-12=7.0 |
| 3 + 5 | 3.1-3.2(m) | |
| 4 + 6 | 3.2-3.3(m) | |
| 17a | 3.44(m) | |
| 17b | 3.61(d broad) | 2J17a-17b=11.5 |
| 12 | 4.96(ddd) | 3J12-21a=7.5; 3J12-21b=6.0; |
| | | 3J12-13=1.2 |
| 14, 15, 16, 18 | 4.49, 5.08, 5.16, 5.41 | |
| 2 | 4.81(d) | 3J2-3=7.5 |

For norcornicinine: the NMR spectrometry data obtained are shown in Tables 3 and 4 hereunder.

TABLE 3

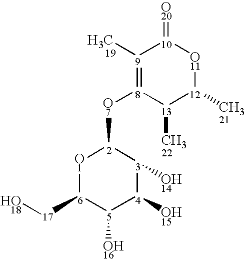

| Proton (D₂O) | δ (ppm) | J (Hz) |
|---|---|---|
| 22 | 1.26 (3H, d) | 3J22 − 13 = 7 |
| 21 | 1.35 (3H, d) | 3J21 − 12 = 6.5 |
| 19 | 1.73 (3H, s) | |
| 13 | 2.80 (1H, q) | 3J13 − 22 = 6.5 |
| 3 + 4 + 5 + 6 | 3.4-3.9 (6H, m) | |
| 12 | 4.56 (1H, q) | 3J12 − 21 = 6.5 |
| 2 | 5.07 (1H, d) | 3J2 − 3 = 6.5 |

TABLE 4

| Carbon (D₂O) | δ (ppm) |
|---|---|
| 19 | 8.01 |
| 22 | 17.06 |
| 21 | 18.182 |
| 13 | 33.175 |
| 17 | 60.14 |
| 3, 4, 5 or 6 | 69.03 |
| 3, 4, 5 or 6 | 72.56 |
| 3, 4, 5 or 6 | 75.25 |
| 3, 4, 5 or 6 | 76.90 |
| 12 | 78.56 |
| 2 | 97.23 |
| 9 | 104.64 |
| 8 | 168.68 |
| 10 | 170.02 |

For X-cornicinine the NMR spectrometry data obtained are shown in Tables 5 and 6 hereunder.

TABLE 5

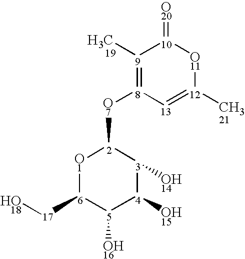

| Proton (D₂O) | δ (ppm) | J (Hz) |
|---|---|---|
| 19 | 1.87 (3H, s) | |
| 21 | 2.26 (3H, s) | |
| 3, 4, 5, 6, 17 | 3.4-3.9 (6H, m) | |
| 2 | 5.19 (¹H, d) | 3J2 − 3 = 6.5 |
| 13 | 6.38 (1H, s) | |

TABLE 6

| Carbone | δ (ppm) |
|---|---|
| 19 | 7.59 |
| 21 | 18.99 |
| 17 | 60.26 |
| 3, 4, 5 or 6 | 69.04 |
| 3, 4, 5 or 6 | 72.42 |
| 3, 4, 5 or 6 | 75.21 |
| 3, 4, 5 or 6 | 76.29 |
| 13 | 97.75 |
| 2 | 98.51 |
| 9 | 102.63 |
| 12 | 162.05 |
| 8 | 165.69 |
| 10 | 169.03 |

The glycosylated triketide δ-lactones such as cornicinine, norcornicinine, and X-cornicinine described herein are novel compounds.

Compounds of formula (1) can be synthesized by manipulated type I polyketide synthases from prokaryotic origins.

Triketide δ-lactone compounds of the present invention can be involved in the synthesis or biosynthesis of a high diversity of secondary metabolites with very high therapeutic potential, and have themselves biological activities.

Glycosylated triketide δ-lactones described herein have a clear biostatic action. Cornicinine and derivatives thereof can also be good tools for studying the fundamental process of cell differentiation.

### Example 3

Non-limiting examples of compounds of formula (2) and (3) according to the present invention are listed hereunder. All stereoisomers of the inventive compounds (2) and (3) are included within the scope of the invention, as pure compounds as well as mixtures thereof. Individual enantiomers, diastereomers, geometric isomers, and combinations and mixtures thereof are all encompassed by the present invention.

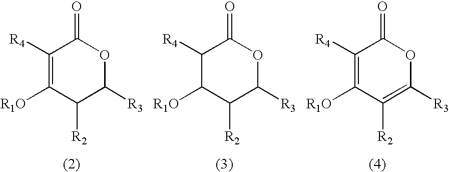

| R₁ | R₂ | R₃ | R₄ |
|---|---|---|---|
| β-D-glucopyranosyl | H | H | H |
| β-D-glucopyranosyl | H | Me | H |
| β-D-glucopyranosyl | H | H | Me |
| β-D-glucopyranosyl | H | Me | Me |
| β-D-glucopyranosyl | Me | H | H |
| β-D-glucopyranosyl | H | H | Et |
| β-D-glucopyranosyl | H | Me | Et |
| β-D-glucopyranosyl | Me | H | Et |
| β-D-glucopyranosyl | Me | Me | Et |
| β-D-glucopyranosyl | Me | H | Me |
| β-D-glucopyranosyl | Me | Me | H |
| β-D-glucopyranosyl | Me | Me | Me |
| β-D-glucopyranosyl | H | H | Pr |
| β-D-glucopyranosyl | H | Me | Pr |
| β-D-glucopyranosyl | Me | H | Pr |
| β-D-glucopyranosyl | Me | Me | Pr |
| galactopyranosyl | H | H | H |
| galactopyranosyl | H | Me | H |
| galactopyranosyl | H | H | Me |
| galactopyranosyl | H | Me | Me |
| galactopyranosyl | Me | H | H |
| galactopyranosyl | H | H | Et |
| galactopyranosyl | H | Me | Et |
| galactopyranosyl | Me | H | Et |
| galactopyranosyl | Me | Me | Et |
| galactopyranosyl | Me | H | Me |
| galactopyranosyl | Me | Me | H |
| galactopyranosyl | Me | Me | Me |
| galactopyranosyl | H | H | Pr |
| galactopyranosyl | H | Me | Pr |
| galactopyranosyl | Me | H | Pr |
| galactopyranosyl | Me | Me | Pr |
| mannopyranosyl | H | H | H |
| mannopyranosyl | H | Me | H |
| mannopyranosyl | H | H | Me |
| mannopyranosyl | H | Me | Me |
| mannopyranosyl | Me | H | H |
| mannopyranosyl | H | H | Et |
| mannopyranosyl | H | Me | Et |
| mannopyranosyl | Me | H | Et |
| mannopyranosyl | Me | Me | Et |
| mannopyranosyl | Me | H | Me |
| mannopyranosyl | Me | Me | H |
| mannopyranosyl | Me | Me | Me |
| mannopyranosyl | H | H | Pr |
| mannopyranosyl | H | Me | Pr |
| mannopyranosyl | Me | H | Pr |
| mannopyranosyl | Me | Me | Pr |
| xylopyranosyl | H | H | H |
| xylopyranosyl | H | Me | H |
| xylopyranosyl | H | H | Me |
| xylopyranosyl | H | Me | Me |
| xylopyranosyl | Me | H | H |
| xylopyranosyl | H | H | Et |
| xylopyranosyl | H | Me | Et |
| xylopyranosyl | Me | H | Et |
| xylopyranosyl | Me | Me | Et |
| xylopyranosyl | Me | H | Me |
| xylopyranosyl | Me | Me | H |
| xylopyranosyl | Me | Me | Me |
| xylopyranosyl | H | H | Pr |
| xylopyranosyl | H | Me | Pr |
| xylopyranosyl | Me | H | Pr |
| xylopyranosyl | Me | Me | Pr |
| cellobiosyl | H | H | H |
| cellobiosyl | H | Me | H |
| cellobiosyl | H | H | Me |
| cellobiosyl | H | Me | Me |
| cellobiosyl | Me | H | H |
| cellobiosyl | H | H | Et |
| cellobiosyl | H | Me | Et |
| cellobiosyl | Me | H | Et |
| cellobiosyl | Me | Me | Et |
| cellobiosyl | Me | H | Me |
| cellobiosyl | Me | Me | H |
| cellobiosyl | Me | Me | Me |
| cellobiosyl | H | H | Pr |
| cellobiosyl | H | Me | Pr |
| cellobiosyl | Me | H | Pr |
| cellobiosyl | Me | Me | Pr |
| lactosyl | H | H | H |
| lactosyl | H | Me | H |
| lactosyl | H | H | Me |
| lactosyl | H | Me | Me |
| lactosyl | Me | H | H |
| lactosyl | H | H | Et |
| lactosyl | H | Me | Et |
| lactosyl | Me | H | Et |
| lactosyl | Me | Me | Et |
| lactosyl | Me | H | Me |
| lactosyl | Me | Me | H |
| lactosyl | Me | Me | Me |
| lactosyl | H | H | Pr |
| lactosyl | H | Me | Pr |
| lactosyl | Me | H | Pr |
| lactosyl | Me | Me | Pr |
| glucofuranosyl | H | H | H |
| glucofuranosyl | H | Me | H |
| glucofuranosyl | H | H | Me |
| glucofuranosyl | H | Me | Me |
| glucofuranosyl | Me | H | H |
| glucofuranosyl | H | H | Et |
| glucofuranosyl | H | Me | Et |
| glucofuranosyl | Me | H | Et |
| glucofuranosyl | Me | Me | Et |
| glucofuranosyl | Me | H | Me |
| glucofuranosyl | Me | Me | H |
| glucofuranosyl | Me | Me | Me |
| glucofuranosyl | H | H | Pr |
| glucofuranosyl | H | Me | Pr |
| glucofuranosyl | Me | H | Pr |
| glucofuranosyl | Me | Me | Pr |
| maltosyl | H | H | H |
| maltosyl | H | Me | H |
| maltosyl | H | H | Me |
| maltosyl | H | Me | Me |
| maltosyl | Me | H | H |
| maltosyl | H | H | Et |
| maltosyl | H | Me | Et |
| maltosyl | Me | H | Et |
| maltosyl | Me | Me | Et |
| maltosyl | Me | H | Me |
| maltosyl | Me | Me | H |
| maltosyl | Me | Me | Me |
| maltosyl | H | H | Pr |
| maltosyl | H | Me | Pr |
| maltosyl | Me | H | Pr |
| maltosyl | Me | Me | Pr |
| 2-amino-1,3-cyclohexanediol | H | H | H |
| 2-amino-1,3-cyclohexanediol | H | Me | H |
| 2-amino-1,3-cyclohexanediol | H | H | Me |
| 2-amino-1,3-cyclohexanediol | H | Me | Me |
| 2-amino-1,3-cyclohexanediol | Me | H | H |
| 2-amino-1,3-cyclohexanediol | H | H | Et |
| 2-amino-1,3-cyclohexanediol | H | Me | Et |
| 2-amino-1,3-cyclohexanediol | Me | H | Et |
| 2-amino-1,3-cyclohexanediol | Me | Me | Et |
| 2-amino-1,3-cyclohexanediol | Me | H | Me |
| 2-amino-1,3-cyclohexanediol | Me | Me | H |
| 2-amino-1,3-cyclohexanediol | Me | Me | Me |
| 2-amino-1,3-cyclohexanediol | H | H | Pr |
| 2-amino-1,3-cyclohexanediol | H | Me | Pr |
| 2-amino-1,3-cyclohexanediol | Me | H | Pr |
| 2-amino-1,3-cyclohexanediol | Me | Me | Pr |

1. A compound of formula (1) and salts, stereoisomeric forms, racemic mixtures, prodrugs, esters and metabolites thereof, wherein 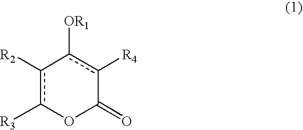
R₁ is a glycosyl moiety, hydroxyl-protected acetate derivatives thereof or amino derivatives thereof;
R₂, R₃ and R₄ are, each independently, selected from the group consisting of hydrogen, C₁₋₆alkyl, C₂₋₂₀alkenyl, C₆₋₂₀arylalkyl, C₃₋₇cycloalkyl, C₃₋₇cycloalkylC₁₋₆alkyl, C₆₋₂₀aryl aralkyl, akylcarbonyloxy, arylcarbonyloxy, alkyloxy, alklylthio, alkylamino, alkyloxyalkyl, alkanoyl, alkylcarbonylalkyl, optionally substituted by one or more substituents independently selected from the group consisting of alkyl, aralkyl, aryl, cycloalkyl, alkyloxycarbonyl, carboxyl, aminocarbonyl, hydroxy, cyano, halogen or amino optionally mono- or disubstituted wherein the substituents are independently selected from the group consisting of alkyl, aryl, aralkyl, aryloxy, arylamino, aryloxyalkyl, arylaminoalkyl, aralkoxy, alkoxy.

2. The compound according to claim 1, wherein said glycosyl moiety is a saccharyl moiety, a hydroxy-substituted cyclohexyl moiety, hydroxyl-protected acetate derivatives thereof or amino derivatives thereof.

3. The compound according to claim 1, wherein R₂, R₃ and R₄ are, each independently, selected from the group consisting of hydrogen, C₁₋₆alkyl, C₂₋₂₀alkenyl, C₆₋₂₀arylalkyl, C₃₋₇cycloalkyl, C₃₋₇cycloalkyC₁₋₆alkyl, C₆₋₂₀aryl and R₁ is selected from the group consisting of glucopyranosyl, fructosyl, galactopyranosyl, mannopyranosyl, ribosyl, ribulosyl, xylulosyl, erythrosyl, threosyl, sorbosyl, psicosyl, tagatosyl, fucosyl, arabinosyl, xylofuranosyl, lyxosyl, talosyl, idosyl, gulosyl, altrosyl, allosyl, mannoheptulosyl, sedoheptulosyl, maltosyl, lactosyl, glucofuranosyl, sucrosyl, cellobiosyl, trehalosyl, gentiobiosyl, melibiosyl, turanosyl, sophorosyl, isosucrosyl, raffinosyl, gentianosyl, 2-amino-2-deoxy glucosyl, 2-amino-2-deoxy galactosyl, 2 amino-1,3-cyclohexanediol, and hydroxyl-protected acetate derivative thereof or amino derivatives thereof.

4. The compound according to claim 1, comprising the formula (2), 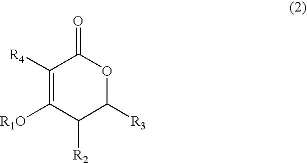
wherein R₁ is a glycosyl moiety, hydroxyl-protected acetate derivatives thereof or amino derivatives thereof;
R₂, R₃ and R₄ are, each independently, selected from the group consisting of hydrogen, C₁₋₆alkyl, C₂₋₂₀alkenyl, C₆₋₂₀arylalkyl, C₃₋₇cycloalkyl, C₃₋₇cycloalkylC₁₋₆alkyl, C₆₋₂₀aryl aralkyl, akylcarbonyloxy, arylcarbonyloxy, alkyloxy, alklylthio, alkylamino, alkyloxyalkyl, alkanoyl, alkylcarbonylalkyl, optionally substituted by one or more substituents independently selected from the group consisting of alkyl, aralkyl, aryl, cycloalkyl, alkyloxycarbonyl, carboxyl, aminocarbonyl, hydroxy, cyano, halogen or amino optionally mono- or disubstituted wherein the substituents are independently selected from the group consisting of alkyl, aryl, aralkyl, aryloxy, arylamino, aryloxyalkyl, arylaminoalkyl, aralkoxy, alkoxy.

5. The compound according to claim 1, comprising the formula (3), 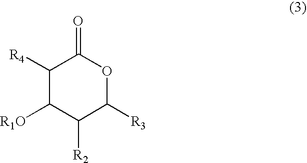
wherein R₁ is a glycosyl moiety, hydroxyl-protected acetate derivatives thereof or amino derivatives thereof;
R₂, R₃ and R₄ are, each independently, selected from the group consisting of hydrogen, C₁₋₆alkyl, C₂₋₂₀alkenyl, C₆₋₂₀arylalkyl, C₃₋₇cycloalkyl, C₃₋₇cycloalkylC₁₋₆alkyl, C₆₋₂₀aryl aralkyl, akylcarbonyloxy, arylcarbonyloxy, alkyloxy, alklylthio, alkylamino, alkyloxyalkyl, alkanoyl, alkylcarbonylalkyl, optionally substituted by one or more substituents independently selected from the group consisting of alkyl, aralkyl, aryl, cycloalkyl, alkyloxycarbonyl, carboxyl, aminocarbonyl, hydroxy, cyano, halogen or amino optionally mono- or disubstituted wherein the substituents are independently selected from the group consisting of alkyl, aryl, aralkyl, aryloxy, arylamino, aryloxyalkyl, arylaminoalkyl, aralkoxy, alkoxy.

6. The compound according to claim 1, comprising the formula (4), 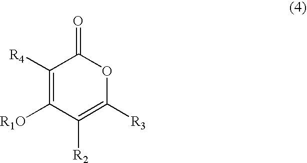
wherein R₁ is a glycosyl moiety, hydroxyl-protected acetate derivatives thereof or amino derivatives thereof;
R₂, R₃ and R₄ are, each independently, selected from the group consisting of hydrogen, C₁₋₆alkyl, C₂₋₂₀alkenyl, C₆₋₂₀arylalkyl, C₃₋₇cycloalkyl, C₃₋₇cycloalkylC₁₋₆alkyl, C₆₋₂₀aryl aralkyl, akylcarbonyloxy, arylcarbonyloxy, alkyloxy, alklylthio, alkylamino, alkyloxyalkyl, alkanoyl, alkylcarbonylalkyl, optionally substituted by one or more substituents independently selected from the group consisting of alkyl, aralkyl, aryl, cycloalkyl, alkyloxycarbonyl, carboxyl, aminocarbonyl, hydroxy, cyano, halogen or amino optionally mono- or disubstituted wherein the substituents are independently selected from the group consisting of alkyl, aryl, aralkyl, aryloxy, arylamino, aryloxyalkyl, arylaminoalkyl, aralkoxy, alkoxy.

7. The compound according to claim 1, wherein R₁ is glucopyranosyl or galactopyranosyl, R₂, R₃ and R₄ are each independently C₁₋₆alkyl.

8. The compound according to claim 1, comprising the formula (5), 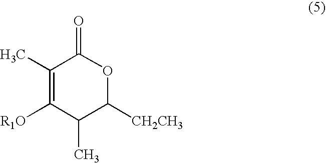
wherein R₁ is selected from the group consisting of glucopyranosyl, fructosyl, galactopyranosyl, mannopyranosyl, ribosyl, ribulosyl, xylulosyl, erythrosyl, threosyl, sorbosyl, psicosyl, tagatosyl, fucosyl, arabinosyl, xylofuranosyl, lyxosyl, talosyl, idosyl, gulosyl, altrosyl, allosyl, mannoheptulosyl, sedoheptulosyl, maltosyl, lactosyl, glucofuranosyl, sucrosyl, cellobiosyl, trehalosyl, gentiobiosyl, melibiosyl, turanosyl, sophorosyl, isosucrosyl, raffinosyl, gentianosyl, 2-amino-2-deoxy glucosyl, 2-amino-2 deoxy galactosyl, 2-amino-1,3-cyclohexanediol, and hydroxyl-protected acetate derivatives thereof or amino derivatives thereof.

9. The compound according to claim 1, comprising the formula (6) 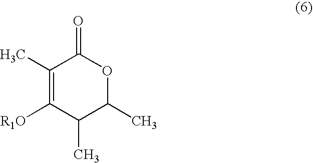
wherein R₁ is selected from the group consisting of glucopyranosyl, fructosyl, galactopyranosyl, mannopyranosyl, ribosyl, ribulosyl, xylulosyl, erythrosyl, threosyl, sorbosyl, psicosyl, tagatosyl, fucosyl, arabinosyl, xylofuranosyl, lyxosyl, talosyl, idosyl, gulosyl, altrosyl, allosyl, mannoheptulosyl, sedoheptulosyl, maltosyl, lactosyl, glucofuranosyl, sucrosyl, cellobiosyl, trehalosyl, gentiobiosyl, melibiosyl, turanosyl, sophorosyl, isosucrosyl, raffinosyl, gentianosyl, 2-amino-2-deoxy glucosyl, 2-amino-2-deoxy galactosyl, 2-amino-1,3-cyclohexanediol, and hydroxyl-protected acetate derivatives thereof or amino derivatives thereof.

10. The compound according to claim 1, comprising the formula (7) 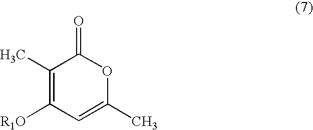
wherein R₁ is selected from the group consisting of glucopyranosyl, fructosyl, galactopyranosyl, mannopyranosyl, ribosyl, ribulosyl, xylulosyl, erythrosyl, threosyl, sorbosyl, psicosyl, tagatosyl, fucosyl, arabinosyl, xylofuranosyl, lyxosyl, talosyl, idosyl, gulosyl, altrosyl, allosyl, mannoheptulosyl, sedoheptulosyl, maltosyl, lactosyl, glucofuranosyl, sucrosyl, cellobiosyl, trehalosyl, gentiobiosyl, melibiosyl, turanosyl, sophorosyl, isosucrosyl, raffinosyl, gentianosyl, 2-amino-2-deoxy glucosyl, 2-amino-2-deoxy galactosyl, 2-amino-1,3-cyclohexanediol, and hydroxyl-protected acetate derivatives thereof or amino derivatives thereof.

11. The compound according to any one of claims 8 to 10, wherein R₁ is β-D-glucopyranosyl.

12. The compound according to claim 1 comprising the formula (8), (9), or (10) or stereoisomers thereof.

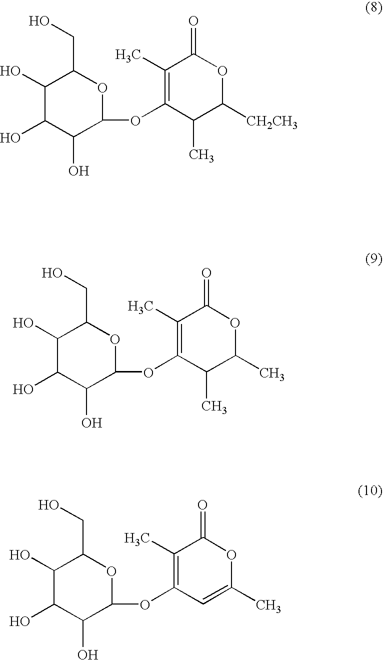

13. The compound according to claim 12 comprising the formula (11), (12) or (13).

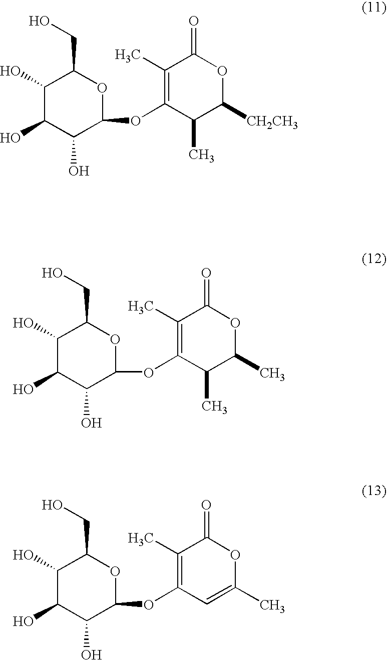

14. A method of disease treatment which comprises administering a composition comprising the compound according to claim 1 to a patient in need thereof.

15. A method of plant treatment which comprises administering a composition comprising the compound according to claim 1 to a plant to be treated.

16. A method of preparing a biostatic agent which comprises blending the compound according to claim 1 with a pharmaceutically acceptable carrier.

17. A method of protecting a surface with a biocide comprising applying a coating composition comprising the compound according to claim 1 as a biocide to the surface to be protected.

18. A method of treating an infection which comprises administering a composition comprising the compound according to claim 1 as an antibiotic to an individual in need thereof.

19. A method of preparing an antifouling agent which comprises blending the compound according to claim 1 with a coating composition.

20. A method of treating an immune disease which comprises administering a composition comprising the compound according to claim 1 as an immunosuppressive agent to an individual in need thereof.

21. A method of treating a fungal disease which comprises administering a composition comprising the compound according to claim 1 as an antifungal to an individual in need thereof.

22. A method of treating cholesterol induced disease which comprises administering a composition comprising the compound according to claim 1 as a cholesterol lowering agent to an individual in need thereof.

23. A method of treating cancer which comprises administering a composition comprising the compound according to claim 1 as an anticancer drug to an individual in need thereof.

24. A pharmaceutical composition comprising at least one compound according to claim 1 and a pharmaceutically acceptable carrier.

25-28. (canceled)

29. A method of synthesizing a polyketide comprising treating a polyketide synthase with a compound according to claim 1 whereby polyketides are synthesized.

30. A method of preparing the compound according to claim 1 which comprises isolation of the compound from Tipulidae spp.
